(19) United States
(12) Patent Application Publication
(10) Pub. No.: US 2012/0071707 A1
(43) Pub. Date: Mar. 22, 2012

(54) MEDICAL DEVICE

(76) Inventor: PETER GINGRAS, Shaker Heights, OH (US)

(21) Appl. No.: 13/087,944

(22) Filed: Apr. 15, 2011

Related U.S. Application Data
(63) Continuation of application No. 11/094,643, filed on Mar. 30, 2005, now abandoned.
(60) Provisional application No. 60/558,067, filed on Mar. 30, 2004.

(51) Int. Cl. A61F 2/04; B23P 11/02

(52) U.S. Cl. 600/30; 29/446

(57) ABSTRACT

A tubular non-woven soft tissue implant has cell patterns. The cell pattern has a continuous circumferential construction with atraumatic edges and a tubular centre. The implant is suitable for stabilising and/or supporting body tissue for example to treat urinary incontinence and/or pelvic floor prolapse.

### CROSS REFERENCE TO RELATED APPLICATIONS

This application is a continuation of U.S. patent application Ser. No. 11/094,643, filed on Mar. 30, 2005, which claims priority to U.S. Provisional Application No. 60/558,067, filed on Mar. 30, 2004, the entire contents of which are hereby incorporated by reference.

### INTRODUCTION

This invention relates generally to a medical device and more specifically to implants that can be used to treat women with stress urinary incontinence (SUI) and pelvic floor prolapse. Urinary incontinence is a serious health concern worldwide. Millions of people suffer from this problem and a pubovaginal sling procedure is a surgical method involving the placement of a sling implant to stabilize or support the bladder neck or urethra.

Slings for treating incontinence may be constructed from synthetic materials such as polypropylene, polytetrafluoroethylene, polyester, and silicone. Slings constructed from non-synthetic materials include allografts, homografts, heterografts, xenografts, autologous tissues, cadaveric fascia, and fascia lata. The supply of non-synthetic slings can vary greatly and certain sizes of non-synthetic materials can be difficult to obtain. For example, autologous material may be difficult or impossible to harvest from some patients due to the health of the patient and the size of the tissue needed for a sling.

The Tension-free Vaginal Tape (TVT) procedure (available from Ethicon, Somerville, N.J., USA) utilizes a nonabsorbable polypropylene mesh. The TVT mesh extends from the rectus fascia in the abdominal region, to a position below the urethra, and back again to the rectus fascia.

### BACKGROUND

Urinary incontinence and pelvic floor prolapse are a major cause of surgery in women and are a major public health challenge and financial burden for most industrialized countries.

There are a variety of different synthetic materials used to treat bodily defects (see U.S. Pat. Nos. 2,671,444; 3,054,406; 3,124,136; 4,452,245; 5,569,273; 6,042,592; 6,090,116; 6,287,316; 6,408,656).

There are a variety of absorbable or partially absorbable materials used to treat bodily defects (see U.S. Pat. Nos. 4,633,873; 4,693,720; 4,838,884, 6,319,264).

There are a variety of implants used to treat urinary incontinence in women (see U.S. Pat. Nos. 4,857,041; 5,840,011; 6,042,534; 6,110,101; 6,306,079; 6,355,065).

The implants used to treat urinary incontinence in women have unique biomechanical characteristics. The properties may be at least partly responsible for the improved clinical success of the implants (see Dietz et al., International Urogynecology Journal and Pelvic Floor Dysfunction 14(4): 239-243, 2003). The modulus of elasticity in tension, also known as Young's modulus, is the ratio of stress to strain on the loading plane. The mechanical properties of different slings have been characterized (see Pariente, Issues in Women's Health 1(1): 9-12, 2003). Different sling materials have different mechanical properties with varying elasticity. It has been suggested that different surgical procedures may benefit from different slings.

| | | Maximum | Young's Modulus |
|---|---|---|---|
| Material | Company | Deformation (%) | (MPa) |
| TVT | Gynecare/Ethicon | 94.5 | 4.3 |
| IVS ® | Tyco Healthcare | 31.4 | 42.0 |
| Uretex ® | Sofradim | 61.4 | 5.0 |
| Spare ® | American Medical | 108.2 | 5.4 |
| I-Stop ® | Clmedical | 17.2 | 40.0 |
| Uratape ® | Mentor Porges | 68.0 | 31.7 |

Sling materials are placed using different surgical approaches. Transvaginal mid-urethral slings, transobturator mid-urethral slings, and suprapubic mid-urethral slings have all been used for treating stress urinary incontinence. The different surgical approaches require that sling materials be placed through different tissue structures that may require materials with unique properties. In addition, the material properties of a sling material determine whether a sheath is required for delivery through tissue. More elastic materials require the use of an inelastic sheath to ensure accurate placement of the material through the tissue.

There are a variety of instruments and methods used to treat urinary incontinence in women (see U.S. Pat. Nos. 5,112,344; 5,611,515; 5,637,074; 5,842,478; 5,860,425; 5,899,909; 6,039,686; 6,273,852; 6,406,423; 6,478,727; 6,702,827; WO 2004/017862; WO 02/39890; WO 02/26108).

Although serious complications associated with sling procedures and materials are infrequent, they are well documented. Complications include urethral obstruction, prolonged urinary retention, bladder perforations, damage to surrounding tissue, nerve entrapment, infection, fragmentation, extrusion, early loss of tensile strength, shrinkage, and sling erosion.

Accordingly, there remains a need for implants for treating women with incontinence and pelvic floor prolapse and methods of making those implants.

### SUMMARY

According to the invention there is provided a medical device comprising a portion of biocompatible material for stabilising and/or supporting body tissue of a patient. The invention provides in a preferred case a medical implant device for stabilising and/or supporting the bladder neck, and/or the urethra, and/or the pelvic floor of a patient.

The biocompatible material may be a non-absorbable material. The biocompatible material may be an absorbable material. The biocompatible material may be a tissue-based material.

In one embodiment the biocompatible material comprises polypropylene, polyethylene terephthalate, polytetrafluoroethylene, polyaryletherketone, nylon, fluorinated ethylene propylene, polybutester, or silicone.

In another embodiment the biocompatible material is bioresorbable or biodegradable. The biocompatible material may be at least partially absorbable by the body. The bioresorbable or biodegradable implant will stay in position and support the urethra or pelvic floor over a predetermined time. The biocompatible material may comprise an absorbable polymer or copolymer such as polyglycolic acid (PGA), polylactic acid (PLA), polycaprolactone, polydioxanone or polyhydroxyalkanoate.

The porous biocompatible film may comprise a biological material such as collagen.

In a further embodiment the device comprises means to facilitate coupling of an attachment element to the device. Preferably the portion of the biocompatible material comprises one or more openings for receiving an attachment element. Ideally the device comprises an attachment element to facilitate attachment of the device to body tissue of a patient. The implant may comprise a mechanical means, adhesive, or bioglue to promote attachment. Most preferably the attachment element comprises a suture, and/or a staple, and/or a protrusion, and/or an adhesive.

The material properties of the biocompatible material may be non-uniform across a portion of the biocompatible material. Ideally the tensile strength and modulus of elasticity are optimised along portions of the biocompatible material to reduce clinical complications while facilitating delivery, placement, and implantation of the material. Physical properties of the material should reduce the risk of creating excess tension on the sling, which may lead to irritation and obstructive voiding symptoms and urinary retention. Furthermore, surgically implanting the material in a predictable tension-free manner may preserve the urethral vascular supply and mucosal seal. The biocompatible material of the invention has physical properties that are optimised to reduce clinical complications with the surrounding tissue. Because surgical treatment options, as described above, require that the material have contact with different tissue structures that impart different levels of stress and strain on the material, the ideal material for treating stress urinary incontinence requires varying physical properties.

In one embodiment the biocompatible material has a homogenous composition with a modulus of elasticity that varies along its length and loading plane to produce and implant with variable physical properties.

In one embodiment, the modulus of elasticity of the biocompatible material is less than about 200 MPa, less than about 100 MPa, less than about 50 MPa, less than about 25 MPa, less than about 20 MPa, less than about 15 MPa, less than about 10 MPa, less than about 5 MPa, less than about 2.5 MPa, and less than about 1.0 MPa.

In one preferred case, the device comprises means to maintain the device in position relative to a patient after deployment. Ideally the portion of the biocompatible material is shaped to maintain the device in position relative to the patient after deployment. Most preferably the portion of the biocompatible material comprises means to engage body tissue of the patient to maintain the device in position relative to the patient after deployment. The engagement means may comprise a protrusion. Preferably the engagement means comprises a plurality of protrusions arranged in a wave-like or dimple-like pattern. The protrusion may be provided by thermally or mechanically shaping the engagement portion of the biocompatible material. In one case, a portion of the device not designed to engage body tissue has a relatively low co-efficient of friction.

In another preferred embodiment the device comprises means to distribute the stabilising and/or supporting force exerted by the portion of the biocompatible material. Ideally the portion of the biocompatible material is shaped to distribute the stabilising and/or supporting force exerted. Preferably, the device should not be placed against any abdominal or visceral organ including the bladder or urethra as complications can occur is the device shrinks. Consequently, the portion of the biocompatible material may comprise a recess to receive the urethra of a patient. The biocompatible material may be configured to generally conform to a patient's urethra and pelvic floor, the biocompatible material being configured to extend circumferentially around the urethra.

In one case the portion of the biocompatible material is movable from a delivery configuration to a deployment configuration. Preferably the delivery configuration is of a lower-profile than the deployment configuration. Ideally the device comprises means to support the portion of the biocompatible material in the deployment configuration. Most preferably the support means is of a metallic material. The support means may be of a shape-memory material. Ideally the shape-memory material is Nitinol.

The device can be used to construct implants that are designed to engage the urethra or pelvic floor. The implants can be configured to conform to the tissue in a three-dimensional preshaped configuration. Ideally, the implant is inserted using minimally invasive techniques and instrumentation. The implant can be constructed with an instrument attachment means for mechanical securement between the implant and instrument. The invention also features methods for producing these implants. These methods can include the step of applying a shape memory material, for example an alloy, such as nitinol, to the implant to facilitate sizing, attachment, and implantation.

The overall shape of the implants can vary depending on the size of the individual and the tissue to be repaired. The overall length, width, and shape of the implants of the present invention can be designed to support a certain area In one embodiment of the invention, the implant consists of separate panels that are positioned individually to support the urethra and pelvic floor. The improvements will come without creating a procedure that is too complex.

The device may comprise an implant in a planar or tubular structure. The device in the planar structure may comprise a biocompatible film or fibre with atraumatic and stable construction when placed under a tension load. The device in the tubular structure may comprise a biocompatible film or fibre with atraumatic and stable construction when placed under a tension load. The device in the tubular structure may comprise an inner deployment substrate that is less elastic than the implant to facilitate sheathless delivery.

In one embodiment, the device may have a visual means for monitoring the force applied to the implant such as a graphic indicator or geometry that has certain characteristics under a certain load. The device may comprise means to determine the magnitude and/or direction of a force applied to the portion of the biocompatible material. Preferably the device comprises means to determine by visual inspection the magnitude and/or direction of a force applied to the portion of the biocompatible material. Ideally the geometrical configuration of at least part of the portion of the biocompatible material is alterable responsive to a change in the magnitude and/or direction of a force applied. Alternatively, an instrument for measuring the load can be attached to the implant. The determining means may comprise an instrument to measure the magnitude and/or direction of a force applied.

The present invention features a device that includes biocompatible film or fibre. The implant material for the device has a thickness of less than about 0.015 inches for non-porous films, less than 0.035 inches for microporous films, and less than 0.030 inches for fibre based implants.

In one embodiment of the invention, the portion of the biocompatible material comprises a surgical mesh soft tissue implant. Preferably the surgical mesh comprises means to facilitate tissue ingrowth and/or cellular infiltration. Ideally the surgical mesh is porous. Most preferably the surgical mesh comprises a porous knit structure. In one case the surgical mesh comprises a knit structure of a first configuration and a knit structure of a second configuration, the first configuration having properties different from the second configuration. The finishing treatment of the surgical mesh can vary to impart different physical properties. Ideally the surgical mesh has pores that are greater than 50 micrometers.

The invention provides a method for producing a soft tissue implant, the method comprising: extruding a first biocompatible polymer to form a fibre; forming a mesh fabric from the fibre; heat setting the mesh fabric; forming the soft tissue implant into a three-dimensional structure; cutting the soft tissue implant into a predetermined shape wherein the method may further comprise the optional step of cleaning the implant.

The invention provides a method for producing a soft tissue implant, the method comprising: extruding a first biocompatible polymer to form a fibre; forming a mesh fabric from the fibre; heat setting the mesh fabric; heat treating selected areas of the mesh fabric under varying degrees of tension to anneal the soft tissue implant; forming the soft tissue implant into a three-dimensional structure; cutting the soft tissue implant into a predetermined shape wherein the method may further comprise the optional step of cleaning the implant.

The invention provides a method for producing a soft tissue implant, the method comprising: extruding a first biocompatible polymer to form a fibre; forming a mesh fabric from the fibre; stretching the mesh fabric under a predetermined load; heat setting the mesh fabric; forming the soft tissue implant into a three-dimensional structure; cutting the soft tissue implant into a predetermined shape wherein the method may further comprise the optional step of cleaning the implant.

In one embodiment of the invention, the portion of the biocompatible material comprises a layer of a biocompatible film. Preferably the device comprises means to facilitate tissue ingrowth and/or cellular infiltration. Ideally the layer is porous. Most preferably the layer comprises a plurality of pores arranged into a cell pattern. In one case the layer comprises a plurality of first pores arranged into a first cell pattern and a plurality of second pores arranged into a second cell pattern, the spacing of the first cell pattern being different from the spacing of the second cell pattern. The spacing between the cell patterns can vary to impart different physical properties. Ideally the diameter of the pores is greater than 50 micrometers.

A given implant can include more than one film (e.g., more than one porous biocompatible film); for example, the invention features an implant that includes a first porous biocompatible film and a second porous biocompatible film, the thickness of the implant being less than about 0.015 inches. The implants, including the materials from which they are made and the cell patterns they can contain are described further below.

The implant is produced by processing a biocompatible polymer into a film and forming pores in the film. In alternative embodiments, the film can be stretched or otherwise manipulated (e.g., trimmed, shaped, washed or otherwise treated) before or after forming pores in the film. Where the implant contains more than one film, the methods of the invention can be carried out by extruding a first biocompatible polymer to form a first film, extruding a second biocompatible polymer to form a second film, attaching the first film to the second film to produce an implant, and forming pores in the implant. Alternatively, the pores can be formed before the two films are adhered to one another. In that instance, the method of making the implant can be carried out by: extruding a first biocompatible polymer to form a first film; forming pores in the first film; extruding a second biocompatible polymer to form a second film; forming pores in the second film; and attaching the first film to the second film to produce an implant.

Where a film is obtained, rather than made, the methods of making the implant can simply require providing a given film that is then attached (e.g., reversibly or irreversibly bound by mechanical or chemical forces), if desired, to another film and/or processed to include one or more pores of a given size and arrangement. The single provided film (or adherent multiple films) can then be subjected to a process (e.g., laser ablation, die punching, or the like) that forms pores within the film(s). Accordingly, any of the methods of the invention can be carried out by providing a given biocompatible film, rather than by producing it by an extrusion or extrusion-like process.

Preferably, the implants of the invention will include (or consist of) a film that has a low profile (or reduced wall thickness) and that is biocompatible. A biocompatible film is one that can, for example, reside next to biological tissue without harming the tissue to any appreciable extent. As noted above, the film(s) used in the implants of the invention can have pores (e.g., open passages from one surface of the film to another) that permit tissue ingrowth and/or cellular infiltration.

The implants of the present invention offer a combination of high porosity, high strength, and low material content, and they may have one or more of the following advantages. They can include pores or porous structures that stimulate fibrosis and reduce inflammation; they can reduce the risk of erosion and formation of adhesions with adjacent tissue (this is especially true with implants having a smooth surface) and atraumatic (e.g., smooth, tapered, or rounded edges); they can simulate the physical properties of the tissue being repaired or replaced, which is expected to promote more complete healing and minimise patient discomfort; their surface areas can be reduced relative to prior art devices (having a reduced amount of material may decrease the likelihood of an immune or inflammatory response). Moreover, implants with a reduced profile can be produced and implanted in a minimally invasive fashion; as they are pliable, they can be placed or implanted through smaller surgical incisions. The methods of the invention may also produce implants with improved optical properties (e.g., implants through which the surgeon can visualise underlying tissue). Practically, the micromachining techniques that can be used to produce the implants of the present invention are efficient and reproducible. The implants described herein should provide enhanced biocompatibility in a low profile configuration while maintaining the requisite strength for the intended purpose.

In one embodiment the biocompatible material has a plurality of cells. The biocompatible material may have a plurality of cells and one or more of the cells in the plurality of cells has a diameter, measured along the longest axis of the cell, of about 10 to about 10,000 microns. The biocompatible material may have a plurality of cells and one or more of the cells of the plurality are essentially square, rectangular, hexagonal, sinusoidal, or diamond-shaped. One or more of the cells of the plurality may be substantially the same shape as the cell shown in FIGS. 3A and 3B.

In one embodiment each of the cells in the plurality of cells has a plurality of undulating elements in the form of a repeating pattern. The undulating elements may be in phase and the force-displacement characteristics are suitable for placement and support. Typically the plurality of cells has a diameter greater than 50 microns and the implant has force displacement characteristics that do not restrict tissue movement.

In one embodiment the implant has cells with the same dimensions that are spaced at different intervals to produce and implant with variable density and physical properties.

In one embodiment, the thickness of the porous biocompatible film is less than about 0.020 inches, less than about 0.019 inches, less than about 0.018 inches, less than about 0.017 inches, less than about 0.016 inches, less than about 0.015 inches, less than about 0.014 inches, less than about 0.013 inches, less than about 0.012 inches, less than about 0.011 inches, less than about 0.010 inches, less than about 0.009 inches, less than about 0.008 inches, less than about 0.007 inches, less than about 0.006 inches, less than about 0.005 inches, less than about 0.004 inches, less than about 0.003 inches, less than about 0.002 inches or is about 0.001 inch.

In a further aspect the invention provides a method for producing an implant, the method comprising: extruding a biocompatible polymer into a film; and forming a plurality of cells in the film; wherein the method may further comprise the optional step of cleaning the implant.

The invention also provides a method for producing an implant, the method comprising: extruding a biocompatible polymer into a film, stretching the film; forming pores in the film to produce a soft tissue implant; wherein the method may further comprise the optional step of cleaning the implant.

The invention further provides a method for producing an implant, the method comprising: extruding a first biocompatible polymer to form a first film; extruding a second biocompatible polymer to form a second film; attaching the first film to the second film to produce an implant; forming pores in the implant; shaping the implant into a configuration; attaching a shape memory element to the implant; wherein the method may further comprise the optional step of cleaning the implant.

In another aspect the invention provides a method for producing a soft tissue implant, the method comprising: extruding a first biocompatible polymer to form a first film; forming pores or cell patterns in the first film; extruding a second biocompatible polymer to form a second film; forming pores in the second film; attaching the first film to the second film to produce a soft tissue implant; wherein the method may further comprise the optional step of cleaning the implant.

According to the invention there is provided an implant for treating urinary incontinence and/or pelvic floor prolapse comprising an elongate tubular sling structure for stabilising and/or supporting body tissue.

In one embodiment of the invention at least part of the sling structure has a substantially reduced elasticity. At least part of the sling structure may have a substantially reduced longitudinal elasticity. The strain of at least part of the sling structure may be less than approximately 10%, when in use.

In one embodiment the elasticity varies along the sling structure. The sling structure may comprise a first portion and a second portion, the first portion having a reduced elasticity relative to the second portion. The first portion may be configured to engage a site of interest of body tissue. The first portion may be configured to engage a urethra of a patient. The second portion may be configured to be positioned in use spaced from a site of interest. The second portion may be configured to dynamically contact body tissue.

In one embodiment the first portion comprises a first element and the second portion comprises a second element, the first element being separate from the second element. The element may be substantially elongate and tubular. The first element may be attached to the second element at an end of the second element. The first element may be attached to the second element at each end of the second element. The first element may be detached from the second element along the length of the second element between the ends. The first element may be located radially inwardly of the second element.

In another case the elasticity varies along the length of the sling structure. The modulus of elasticity may vary by more than 2.5 MPa along the sling structure, or more than 5 MPa, or more than 10 MPa, or more than 15 MPa, or more than 20 MPa, or more than 25 MPa, or more than 50 MPa, or more than 100 MPa, or 200 MPa.

In another embodiment the sling structure is configured to be attached to body tissue. The sling structure may be configured to facilitate coupling of an attachment element to the sling structure. The sling structure may comprise one or more attachment openings for receiving an attachment element. The implant may comprise an attachment element for attaching the sling structure to body tissue. The attachment element may comprise a suture and/or staple and/or an adhesive.

In one case the sling structure is shaped for attachment of the sling structure to body tissue. The sling structure may be three-dimensionally shaped for attachment of the sling structure to body tissue. The sling structure may comprise one or more engagement formations for attaching the sling structure to body tissue. The engagement formation may comprise a protrusion. The sling structure may comprise a plurality of engagement formations arranged in a wave-like pattern. The sling structure may comprise a plurality of engagement formations arranged in a dimple-like pattern. The diameter of the sling structure may vary along the length of the sling structure. The diameter of the sling structure may vary in a step-wise manner along the length of the sling structure. The engagement formation may be provided by the step-wise variation in the diameter of the sling structure.

In another case the engagement formation comprises a recess for receiving a portion of body tissue. The recess may comprise a notch in the sling structure. The recess may be configured to receive at least part of a urethra. The sling structure may be configured to extend circumferentially around at least part of the urethra.

In one embodiment the sling structure comprises an attachment portion configured to be attached to body tissue and a detached portion configured to remain detached from body tissue. The attachment portion may have a relatively high co-efficient of friction. The detached portion may have a relatively low co-efficient of friction.

In another case the external surface of the sling structure, which is contactable with body tissue, is substantially atraumatic. The external surface of the sling structure may be substantially smooth. The external surface of the sling structure may be substantially free of fraying.

In another embodiment the sling structure is of a laminate configuration. The sling structure may comprise a first layer and a second layer. The first layer may be of the same material as the second layer. The first layer may be of a different material to the second layer. The rate of absorption of the first layer may be greater than the rate of absorption of the second layer. The first layer may be of an absorbable biocompatible material and the second layer may be of a non-absorbable biocompatible material.

In one case the first layer is attached to the second layer. The first layer may be bonded to the second layer.

In another case the implant comprises a reinforcement for the sling structure. The sling structure may be of a composite configuration. The implant may comprise one or more fibres to reinforce the sling structure. The fibre may be substantially non-elastic. The fibre may be woven into the sling structure. The fibre may be attached to a surface of the sling structure.

In another embodiment the sling structure is configured to facilitate tissue ingrowth and/or cellular infiltration. The sling structure may be at least partially porous. The sling structure may comprise a plurality of pores arranged into a cell pattern. The cell pattern may vary across the sling structure. The pore size may remain substantially constant across the sling structure, and the extent of material extending between adjacent pores may vary across the sling structure. The density of the cell pattern may vary along the sling structure. At least one of the pores may be substantially hexagonally shaped. The diameter of at least one of the pores may be greater than 50 micrometers.

In another case the sling structure comprises a non-woven biocompatible material. The non-woven biocompatible material may comprise a film material. The film material may be non-porous. The thickness of the film material may be less than 0.015 inches. The film material may be microporous. The thickness of the film material may be less than 0.035 inches.

In another embodiment the sling structure comprises one or more biocompatible fibres. The fibre may comprise a monofilament fibre. The thickness of the fibre may be less than 0.030 inches. The fibres may be woven to form a mesh. The fibres may be knitted to form a mesh. The mesh may include stitch loop intersections.

In one embodiment the sling structure comprises an absorbable biocompatible material. The sling structure may comprise a non-absorbable biocompatible material. The sling structure may comprise a tissue-based biocompatible material.

In one case at least some of the mechanical properties of the mesh are substantially omnidirectional. The elasticity of the mesh may be substantially omnidirectional.

The implant may be configured to distribute the stabilising and/or supporting force. The sling structure may be shaped to distribute the stabilising and/or supporting force exerted.

The sling structure may comprise a recess to receive at least part of the urethra of a patient.

In one embodiment the sling structure is movable from a delivery configuration to a deployment configuration. The delivery configuration may be of a lower-profile than the deployment configuration. The implant may comprise a support to support the sling structure in the deployment configuration. The support may be of a metallic material. The support may be of a shape-memory material. The shape-memory material may be Nitinol.

In another embodiment the implant comprises an indicator to determine the magnitude and/or direction of a force applied to the sling structure. The indicator may be configured to determine by visual inspection the magnitude and/or direction of a force applied to the sling structure. The geometrical configuration of at least part of the sling structure may be alterable responsive to a change in the magnitude and/or direction of a force applied. The indicator may comprise an instrument to measure the magnitude and/or direction of a force applied.

In another case the sling structure is configured to stabilise and/or support a bladder neck. The sling structure may be configured to stabilise and/or support a urethra. The sling structure may be configured to stabilise and/or support a pelvic floor.

The invention also provides in another aspect an implant for treating urinary incontinence and/or pelvic floor prolapse comprising a structure for stabilising and/or supporting body tissue, at least part of the structure having a substantially reduced elasticity.

The structure may be substantially planar.

In a further aspect, the invention provides an implant for treating urinary incontinence and/or pelvic floor prolapse comprising a structure for stabilising and/or supporting body tissue, the structure being shaped for attachment of the structure to body tissue.

The invention provides in a further aspect an implant for treating urinary incontinence and/or pelvic floor prolapse comprising a structure for stabilising and/or supporting body tissue, the structure comprising a non-woven biocompatible material.

In another aspect of the invention there is provided an implant for treating urinary incontinence and/or pelvic floor prolapse comprising a structure for stabilising and/or supporting body tissue, the structure being shaped to distribute the stabilising and/or supporting force exerted.

The invention also provides in another aspect a method of producing an implant for treating urinary incontinence and/or pelvic floor prolapse, the method comprising the step of forming a structure for stabilising and/or supporting body tissue.

In one case implant comprises an implant of the invention. The method may comprise the step of treating the structure to reduce the elasticity of at least part of the structure. The elasticity may be reduced by heat treating the structure under tension. The elasticity may be reduced by heat treating the structure under vacuum. A first part of the structure may be treated to reduce the elasticity of the first part, and a second part of the structure may remain untreated without any reduction in the elasticity of the second part. The method may comprise the step of treating the structure to make at least some of the mechanical properties of the structure substantially omnidirectional. The mesh may be stretched in a first direction while holding the mesh in a second direction perpendicular to the first direction.

In another aspect of the invention there is provided a medical instrument for delivering an implant of the invention to a desired site and for deploying the implant at the desired site.

The invention also provides a method of treating urinary incontinence and/or pelvic floor prolapse comprising the steps of:
delivering the implant of the invention to a desired site; and
deploying the implant at the desired site.

According to another aspect of the invention there is provided a soft tissue implant comprising a non-woven biocompatible material, the implant being at least partially porous with a plurality of pores arranged into a cell pattern, the cell pattern varying across the implant.

The invention also provides in another aspect a soft tissue implant comprising a non-woven biocompatible material, the mechanical properties of the implant varying across the implant.

In a further aspect of the invention, there is provided a soft tissue implant comprising a non-woven biocompatible material, the implant being shaped for attachment of the implant to body tissue.

In another aspect, the invention provides a soft tissue implant comprising a non-woven biocompatible material, the implant being shaped to distribute the force exerted by the implant on body tissue.

The invention also provides in another aspect a soft tissue implant comprising a non-woven biocompatible material, and a reinforcement to reinforce an edge region of the material.

In one case the reinforcement is configured to increase the strength of the edge region. The reinforcement may comprise an inelastic element.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

The invention will be more clearly understood from the following description of some embodiments thereof, given by way of example only, with reference to the accompanying drawings, in which:—

FIGS. 1A-1C are scanning electron micrographs of a polypropylene surgical mesh made as described in Example 2, at 35×, 80×, and 70×, respectively.

FIG. 1D is a force displacement graph for the polypropylene surgical mesh samples made as described in Example 3.

FIGS. 2A-2B are scanning electron micrographs of a polypropylene mesh made as described in Example 1, at 35×.

FIG. 2C is a perspective view of a tubular soft tissue implant sling.

FIG. 2D is a perspective view of a tubular nonwoven soft tissue implant sling.

FIG. 2E is a perspective view of a tubular nonwoven soft tissue implant sling with instrument attachment means.

FIG. 2F is a perspective view of a tubular nonwoven soft tissue implant sling with instrument attachment means and inner delivery substrate.

FIGS. 3A-3B are perspective views of nonwoven soft tissue implant materials.

FIG. 3C is a perspective view of a nonwoven soft tissue implant sling with a combination of cells patterns.

FIG. 3D is a perspective view of a nonwoven soft tissue implant for pelvic floor reconstruction with a combination of cells patterns.

FIG. 3E is a perspective view of a nonwoven soft tissue implant sling with varying cell pattern densities.

FIG. 4A is a perspective view of a soft tissue implant sling with a preshaped section for the urethra.

FIG. 4B is a perspective view of a soft tissue implant sling with a notched preshaped section for tissue attachment.

FIG. 4C is a perspective view of a soft tissue implant sling with a wavelike contoured surface for tissue attachment.

FIG. 4D is a perspective view of a soft tissue implant sling with a dimple-like contoured surface for tissue attachment.

FIGS. 5A-B are a scanning electron micrographs of a nonwoven soft tissue implant sling made as described in Example 4, at 35×.

FIG. 6 is a flow chart illustrating some of the steps in a method of producing an implant for treating stress urinary incontinence or pelvic floor prolapse.

### SELECTED REFERENCE NUMERALS IN DRAWINGS

10 monofilament fibres
12 large pore
14 stitch loop intersections
16 thickness profile
20 tubular surgical mesh soft tissue implant sling
22 tubular surgical mesh soft tissue implant sling edge
24 tubular surgical mesh soft tissue implant sling centre
30 tubular nonwoven soft tissue implant sling
32 1.3 mm cell pattern
34 tubular surgical mesh soft tissue implant sling edge
36 tubular surgical mesh soft tissue implant sling centre
38 instrument attachment means
40 1.3 mm nonwoven soft tissue implant
42 nonwoven film
44 2.6 mm nonwoven soft tissue implant
46 2.6 mm cell pattern
48 1.3 and 2.6 mm nonwoven soft tissue implant
50 low and high density nonwoven soft tissue implant
52 thin strut width
54 thick strut width
60 preshaped implant sling
62 preshaped section
64 notched implant sling
66 notched section
70 high friction waved implant sling
72 wave contoured section
80 high friction dimpled implant sling
82 dimple contoured section
90 0.7 mm nonwoven soft tissue implant
92 0.7 mm cell pattern
94 strut width
96 contoured edge

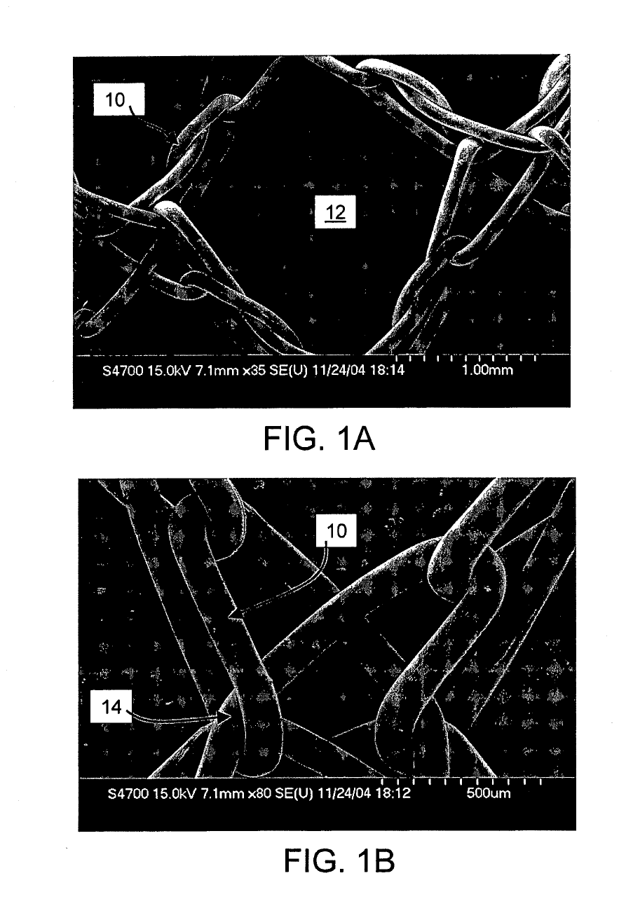

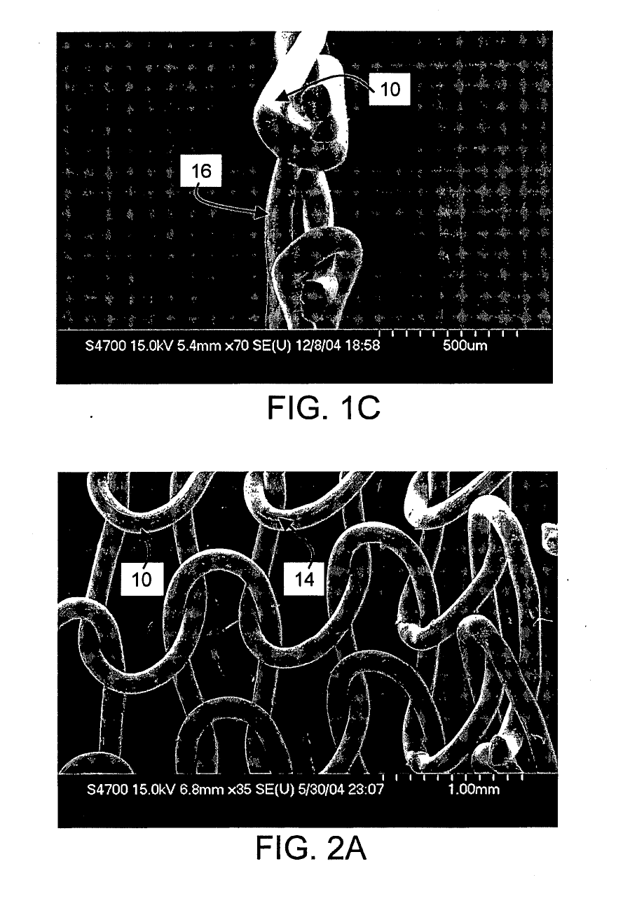

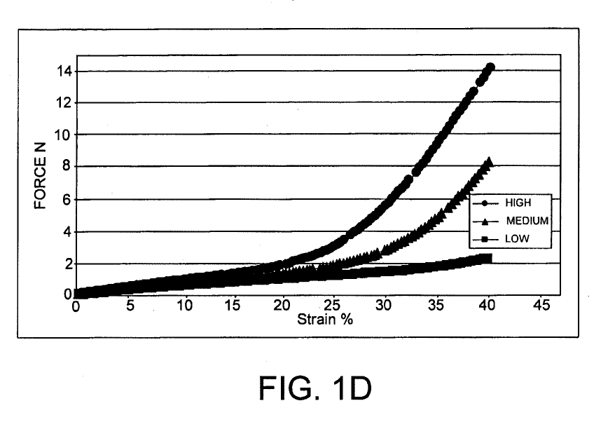

Referring to FIGS. 1A and 1B, scanning electron micrographs of uncondensed polypropylene surgical mesh are shown at 35× and 80×, respectively. Monofilament fibres 10 are used to knit the mesh into a large pore 12 construction which permits tissue ingrowth upon implantation. Stitch loop intersections 14 are created during the knitting process. Referring to FIG. 1C, a scanning electron micrograph of uncondensed polypropylene mesh, the surgical mesh thickness profile 16 is determined by the distance between monofilament fibres 10 from a first side to a second side of the surgical mesh (e.g., from the back to the front).

Referring to FIG. 1D, force displacement graphs for the samples made as described in Example 3 are shown. Surgical mesh samples under low, moderate, and high strain are graphed using force displacement criteria. A higher amount of force is required to displace the high strain sample compared to the low and moderate strain samples.

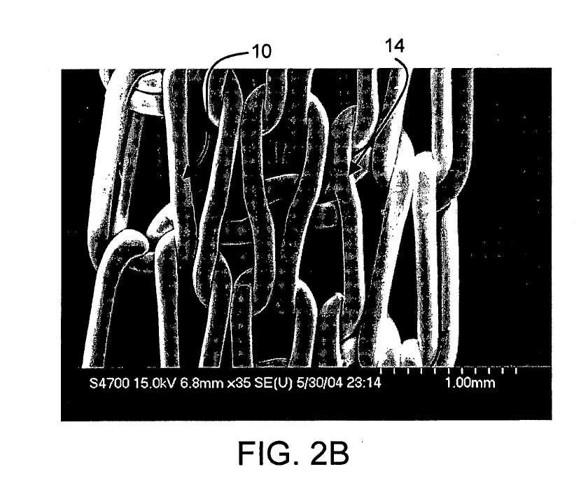

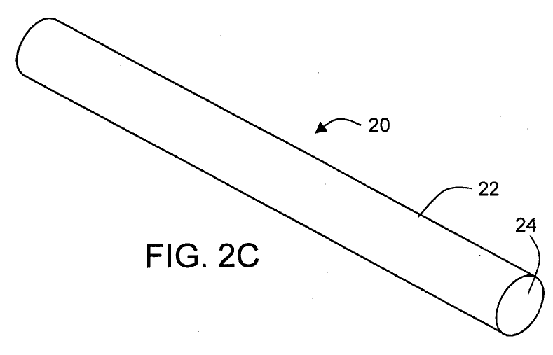

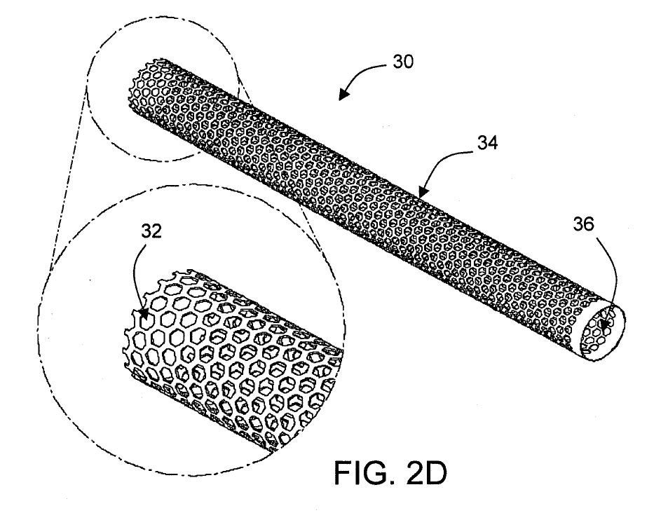

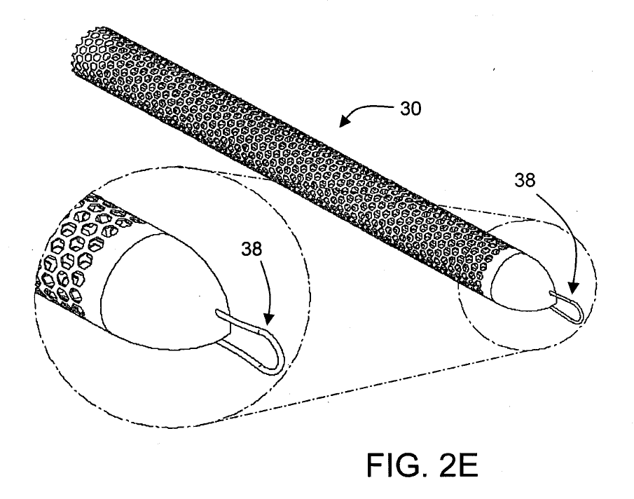

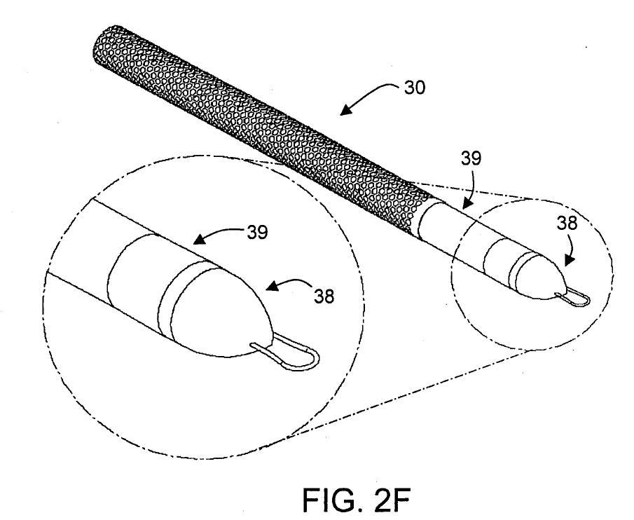

Referring to FIGS. 2A and 2B, scanning electron micrographs of tubular surgical mesh soft tissue implant slings 20 are shown at 35×. Monofilament fibres 10 are used to knit the mesh into a tubular weft knit construction, which permits tissue ingrowth upon implantation. Stitch loop intersections 14 are created during the knitting process.

Referring to FIG. 2C, a perspective view of a tubular soft tissue implant sling 20 is depicted with a continuous circumferential construction with atraumatic edges 22. The tubular centre 24 of the tubular sling is depicted. The tubular soft tissue implant sling can be made from a variety of biocompatible materials including surgical meshes and nonwoven soft tissue implants.

Referring to FIG. 2D, a perspective view of a tubular nonwoven soft tissue implant sling 30 with cells patterns 32. The cell pattern 32 is depicted with a continuous circumferential construction with atraumatic edges 34. The tubular centre 36 of the tubular sling is depicted.

Referring to FIG. 2E is a perspective view of a tubular nonwoven soft tissue implant sling 30 with instrument attachment means 38.

Referring to FIG. 2F is a perspective view of a tubular nonwoven soft tissue implant sling 30 with instrument attachment means 38 and inner delivery substrate 39.

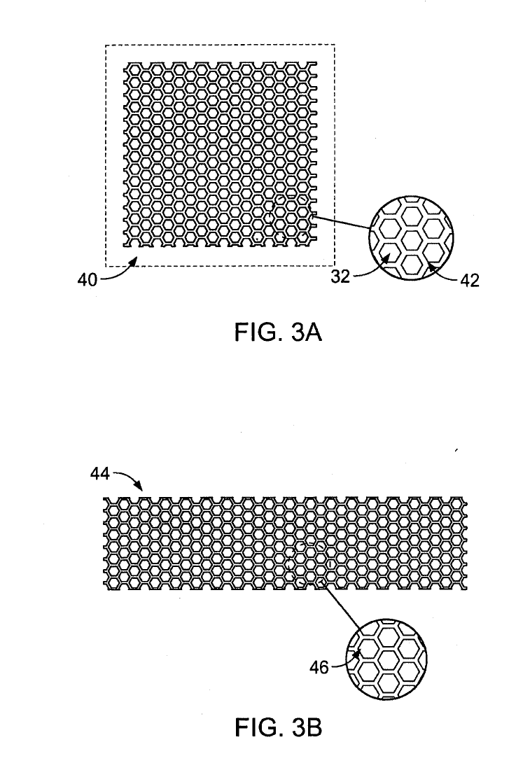

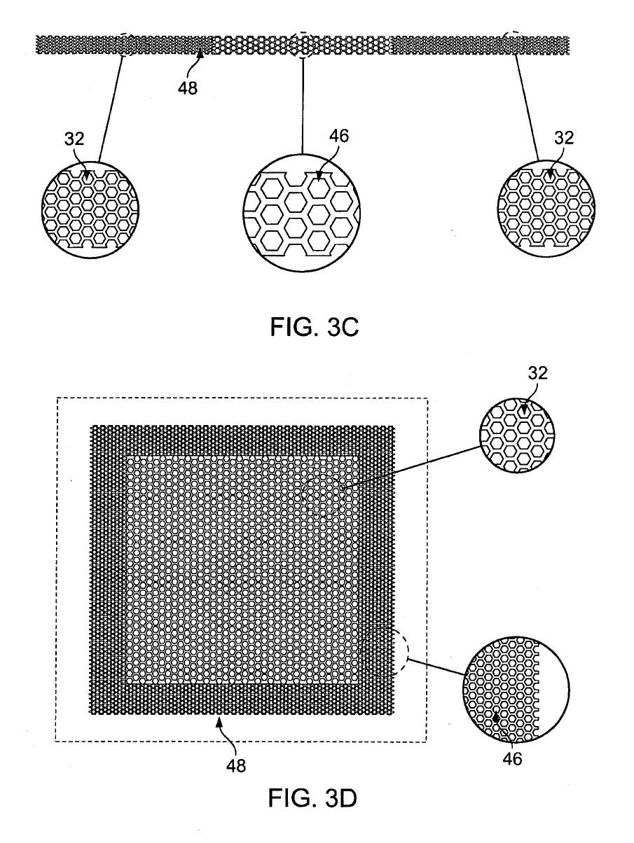

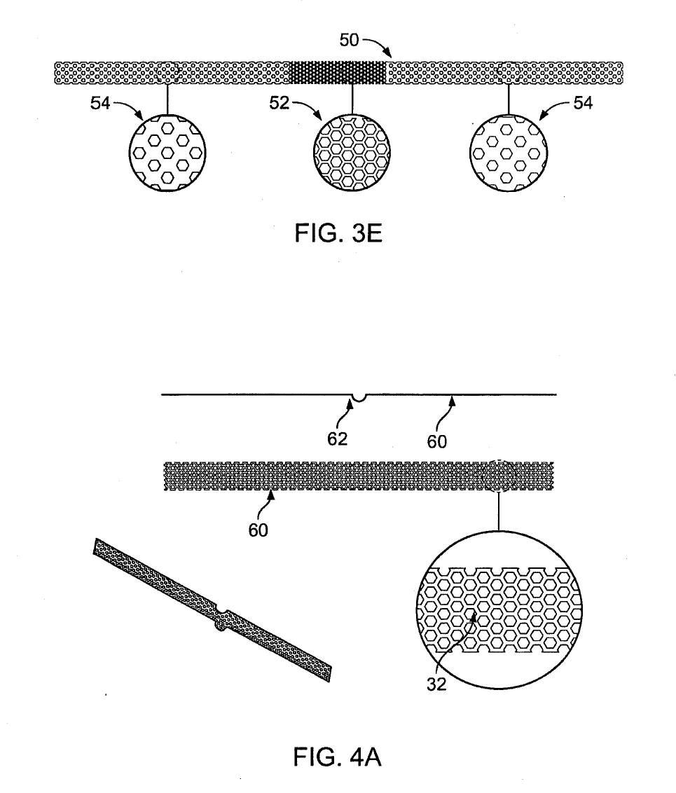

Referring to FIG. 3A is a perspective view of a nonwoven soft tissue implant 40 comprising a 1.3 mm hexagon cell pattern 32. The nonwoven material 42 may be machined to produce the implant. The material illustrated in FIG. 3A is a perspective view of nonwoven biocompatible film 42. The film 42 has known or discernible dimensions (width, length, and thickness), which can be modified or left intact in the manufacture of a tubular nonwoven soft tissue implant sling. In this case the film 42 is a single-layer, smooth-edged film. Film 42 can be a laminate, which can also be used, with or without further modification, to manufacture the implants of the present invention. Multiple layers of biocompatible film 42 can be added together to improve the mechanical properties (e.g., tear resistance and burst strength) of the implant. For example, a first film 42 can be bonded to a second film 42. The bonding may be a thermal bond using hydraulic presses such as those manufactured by Lauffer Pressen (Horb, Germany).

Biocompatible materials useful in film 42 can include non-absorbable polymers such as polypropylene, polyethylene, polyethylene terephthalate, polytetrafluoroethylene, polyaryletherketone, nylon, fluorinated ethylene propylene, polybutester, and silicone, or copolymers thereof (e.g., a copolymer of polypropylene and polyethylene); absorbable polymers such as polyglycolic acid (PGA), polylactic acid (PLA), polycaprolactone, polydioxanone and polyhydroxyalkanoate, or copolymers thereof (e.g., a copolymer of PGA and PLA); or tissue based materials (e.g., collagen or other biological material or tissue obtained from the patient who is to receive the implant or obtained from another person. The polymers can be of the D-isoform, the L-isoform, or a mixture of both. An example of a biocompatible material for producing the laminated film structure 42 is expanded polytetrafluoroethylene.

In the case of a laminate the various layers may be of the same or different materials. For example, in the case of absorbable material the material of the layers may be selected to have varying rates of absorption.

Referring to FIG. 3B is a perspective view of a nonwoven soft tissue implant sling 20 comprising a 2.6 mm hexagon cell pattern 22.

Referring to FIG. 3C, a perspective view of a nonwoven soft tissue implant sling 48 with varying cells patterns along its length. The cell patterns include the 1.3 mm hexagon cell 32 at the ends and the 2.6 mm hexagon cell 46 in the centre.

The implant can have enhanced physical properties along its peripheral edges to improve suture or staple retention strength. The strength of material along the peripheral edges may be higher to improve the physical properties in this region so that sutures do not pull out and cause failure. The material content in these regions can be increased to improve the physical properties. In addition, attachment points can be created along the edge for receiving sutures, staples, or adhesives. The attachment points can be used to attach separate panels to one another to create the implant.

Referring to FIG. 3D, a perspective view of a nonwoven soft tissue implant 48 with a combination of cells patterns. The cell patterns include the 1.3 mm hexagon cell 32 and the 2.6 mm hexagon cell 46. The cell patterns include the 1.3 mm hexagon cell 32 at the edges and the 2.6 mm hexagon cell 46 in the centre.

Referring to FIG. 3E, a perspective view of a nonwoven soft tissue implant sling 50 with a cells pattern of varying density. The cell patterns include the 1.3 mm hexagon cell 32 with thin strut widths 52 in lower density regions and thick strut widths 54 in higher density regions.

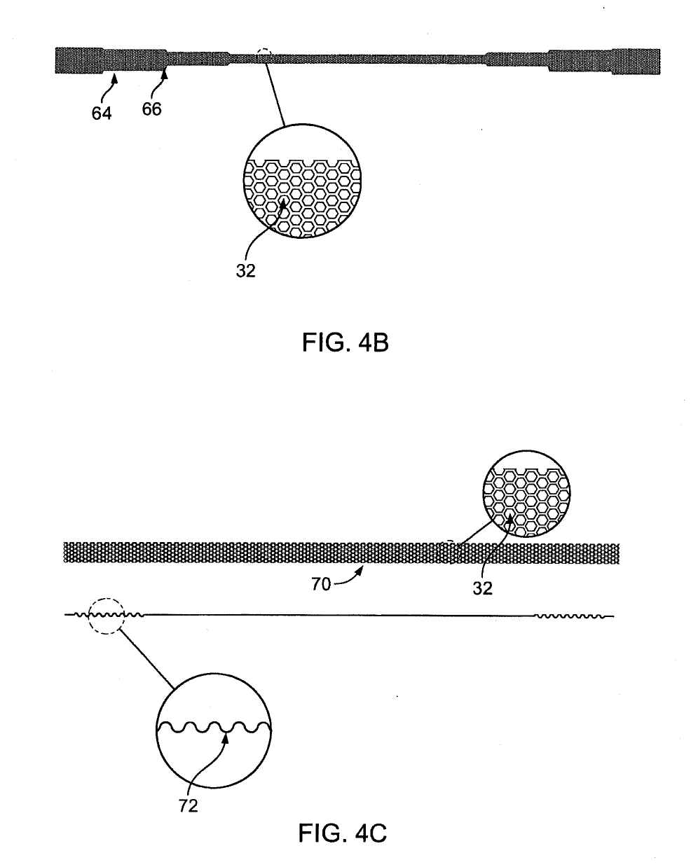

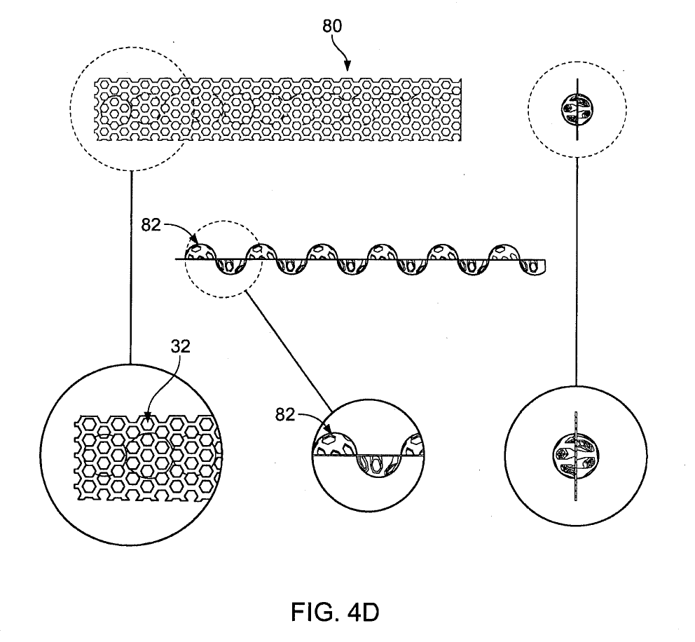

Referring to FIG. 4A, a perspective view of a nonwoven soft tissue implant sling 60 with a preshaped section for the urethra 62. The cell pattern includes the 1.3 mm hexagon cell 32. The preshaped section for the urethra 62 is 5 mm in diameter and is designed to accommodate the urethra so that the force of the soft tissue implant is distributed uniformly around the urethra. This may reduce the risk of erosion of the implant into the urethra. Soft biocompatible materials can also be placed in the preshaped section.

Referring to FIG. 4B, is a perspective view of a nonwoven soft tissue implant sling 64 with a notched preshaped section 66 for tissue attachment. The cell pattern includes the 1.3 mm hexagon cell 32. The notched preshaped section 66 for tissue attachment is designed to prevent the implant from slipping after it is in position.

Referring to FIG. 4C, a perspective view of a nonwoven soft tissue implant sling 70 with a wavelike contoured surface 72 for tissue attachment. The cell pattern includes the 1.3 mm hexagon cell 32. The contoured surface 72 for tissue attachment is designed to prevent the implant from slipping after it is in position.

Referring to FIG. 4D, a perspective view of a nonwoven soft tissue implant sling 80 with a dimple-like contoured surface 82 for tissue attachment. The cell pattern includes the 1.3 mm hexagon cell. The contoured surface 82 for tissue attachment is designed to prevent the implant from slipping after it is in position.

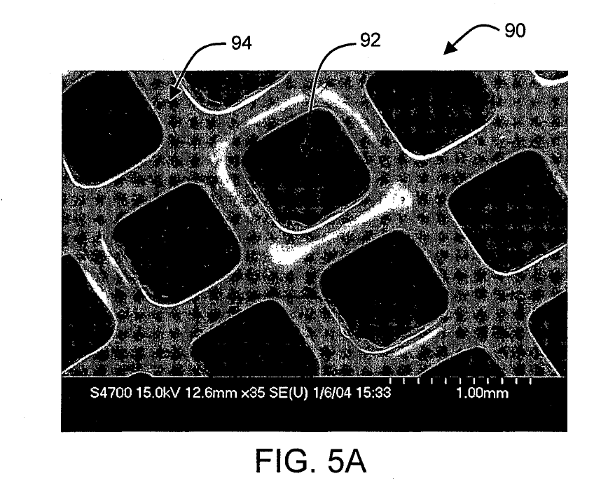

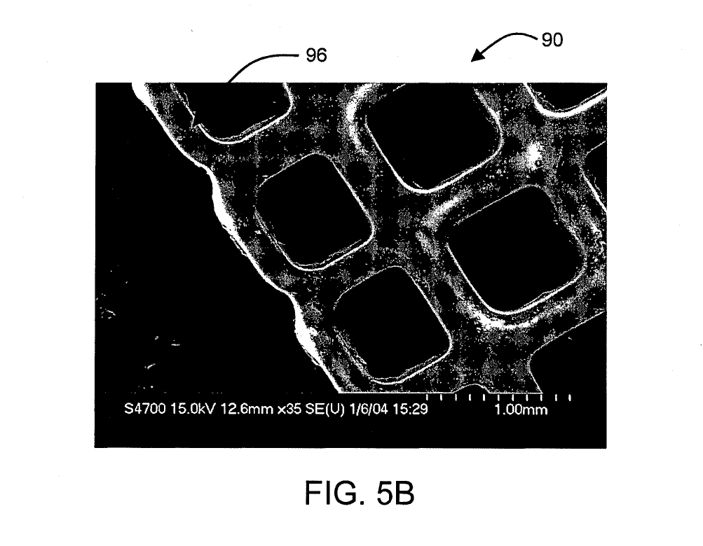

Referring to FIGS. 5A and 5B, scanning electron micrographs of a nonwoven soft tissue implant sling 90 are shown at 35×. A series of 0.7 mm square cells 92 and struts 94 are used to construct the implant. A smooth contoured edge 96 is depicted which provides for easy insertion and reduced trauma at the tissue interface.

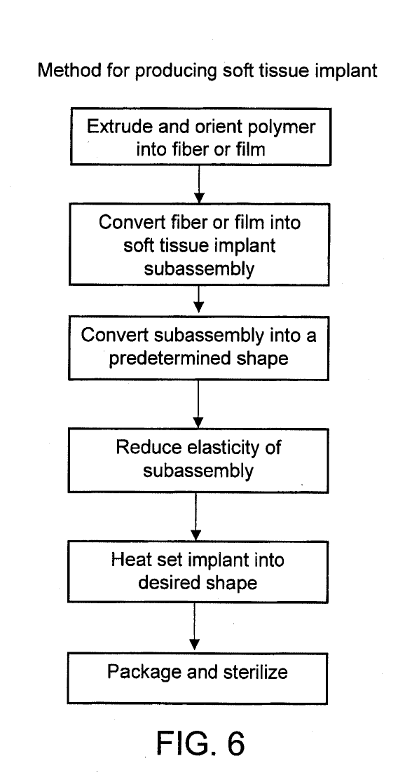

Referring to FIG. 6, a diagram illustrates one embodiment of the present methods. While the methods are described further below, they can include the steps of extruding an orienting a polymer into a fibre or film; converting the fibre or film into a soft tissue implant; heat setting the soft tissue implant; forming the soft tissue implant into a three-dimensional structure (a subassembly); converting the subassembly into a predetermined shape; cleaning the implant; and packaging and sterilizing the implant.

Polytetrafluoroethylene (PTFE) polymer has useful properties as an implant material. PTFE can be processed into a microporous form using an expansion procedure. Bard Vascular Systems (Tempe, Ariz., USA) manufactures ePTFE. Expanded PTFE offers a combination of strength and flexibility together with extensive biocompatibility.

Medical implant applications for the soft tissue implant technology described above may include but are not limited to procedures for treating stress urinary incontinence and pelvic floor prolapse. The soft tissue implant may be produced in a variety of shapes and sizes for the particular indication. One may select a non-absorbable implant for patients that require permanent treatment and long-term durability and strength. Alternatively, one may select an absorbable soft tissue implant for patients that require temporary treatment and tissue remodelling when one wants to avoid the potential complications associated with a permanent implant.

In addition, the soft tissue implant product design may be produced in three-dimensional forms to facilitate sizing. An example is an implant with a curvature to construct a substantially cylindrical shape. A three dimensional structure could be machined using a system incorporating a third axis for micromachining. Alternatively, a substantially two-dimensional soft tissue implant could be thermoformed into a three-dimensional shape after machining.

### EXAMPLES

### Example 1A

We constructed a tubular surgical mesh implant using a 6 mil polypropylene monofilament. A Lamb Model ST3AH/ZA weft knitting machine (Lamb Knitting Machine Corporation, Chicopee, Mass., USA) was used to construct the surgical mesh. Cylinder number 33-62.16 having 8 needles at 7 needles per inch with one end of filament was used to construct the tubular surgical mesh. The implant exhibited fray resistant edges and longitudinal elasticity.

### Example 1B

The tubular surgical mesh from Example 1A assembly was brought under tension to 160° C. under 100 N/cm² and vacuum between two layers of DuPont Kapton 200HN film (Circleville, Ohio, USA) using a Lauffer RLKV 40/1 vacuum lamination press. The implant exhibited a lower elasticity compared to the original tubular surgical mesh assembly.

### Example 2

We constructed a knitted polypropylene surgical mesh implant using 4-mil monofilament polypropylene fibre. The fibre was produced using Marlex HGX-030-01 polypropylene homopolymer. The knitted surgical mesh had elasticity in the machine and transverse directions. A warp knit was employed to give the mesh exceptional tensile strength and to prevent runs and unraveling. A suitable mesh is produced when employing the following pattern wheel or chain drum arrangements: front guide bar, 1-0/1-2/2-3/2-1 and back guide bar, 2-3/2-1/1-0/1-2.

### Examples 3

The surgical mesh constructed in Example 2 was processed in a manner to reduce the elasticity of the surgical mesh implant. The surgical mesh implant was brought to 0% (low), 7.5% (medium), and 15% (high) strain and heat treated at 155° C., under a force of 75 N/cm², and vacuum between two layers of DuPont Kapton 200HN film (Circleville, Ohio, USA) using a Lauffer RLKV 40/1 vacuum lamination press. The surgical mesh had a thickness of 0.023 inches before the pressure heat treatment and a thickness of 0.008 inches after the heat treatment. The surgical mesh processed under strain exhibited a high modulus of elasticity compared to the surgical mesh processed while not under strain.

### Example 4

A nonwoven soft tissue implant was constructed using biaxially oriented polymer films. Expanded PTFE film, part number 1TM22250, was obtained from BHA Technologies (Slater, Mo., USA). Twelve sheets of the film were placed between two sheets of DuPont Kapton 200HN film (Circleville, Ohio, USA). The sheet assembly was brought to 350° C. at 280 N/cm² of constant pressure for 15 minutes using a Lauffer RLKV 40/1 vacuum lamination press. The laminated assembly was machined into square cell patterns using a die punch produced by Elite Tool & Die (Smithstown, Ireland). The nonwoven soft tissue implant exhibited a high modulus of elasticity compared to commercial sling implants.

A number of embodiments of the invention have been described. Nevertheless, it will be understood that various modifications may be made without departing from the spirit and scope of the invention.

The invention is not limited to the embodiments hereinbefore described, with reference to the accompanying drawings, which may be varied in construction and detail.

1-56. (canceled)

57. An implant for treating urinary incontinence and/or pelvic floor prolapse comprising a structure for stabilising and/or supporting body tissue, at least part of the structure having a substantially reduced elasticity.

58. An implant as claimed in claim 57 wherein the structure is substantially planar.

59. An implant for treating urinary incontinence and/or pelvic floor prolapse comprising a structure for stabilising and/or supporting body tissue, the structure being shaped for attachment of the structure to body tissue.

60. An implant for treating urinary incontinence and/or pelvic floor prolapse comprising a structure for stabilising and/or supporting body tissue, the structure comprising a non-woven biocompatible material.

61. An implant for treating urinary incontinence and/or pelvic floor prolapse comprising a structure for stabilising and/or supporting body tissue, the structure being shaped to distribute the stabilising and/or supporting force exerted.

62. A method of producing an implant for treating urinary incontinence and/or pelvic floor prolapse, the method comprising the step of forming a structure for stabilising and/or supporting body tissue.

63. (canceled)

64. A method as claimed in claim 62 wherein the method comprises the step of treating the structure to reduce the elasticity of at least part of the structure.

65. A method as claimed in claim 64 wherein a first part of the structure is treated to reduce the elasticity of the first part, and a second part of the structure remains untreated without any reduction in the elasticity of the second part.

66. A method as claimed in claim 62 wherein the method comprises the step of treating the structure to make at least some of the mechanical properties of the structure substantially omnidirectional.

67. A method as claimed in claim 66 wherein the mesh is stretched in a first direction while holding the mesh in a second direction perpendicular to the first direction.

68-69. (canceled)

70. A soft tissue implant comprising a non-woven biocompatible material, the implant being at least partially porous with a plurality of pores arranged into a cell pattern, the cell pattern varying across the implant.

71. A soft tissue implant comprising a non-woven biocompatible material, the mechanical properties of the implant varying across the implant.

72. A soft tissue implant comprising a non-woven biocompatible material, the implant being shaped for attachment of the implant to body tissue.

73. A soft tissue implant comprising a non-woven biocompatible material, the implant being shaped to distribute the force exerted by the implant on body tissue.

74. A soft tissue implant comprising a non-woven biocompatible material, and a reinforcement to reinforce an edge region of the material.

75. An implant as claimed in claim 74 wherein the reinforcement is configured to increase the strength of the edge region.

76. An implant as claimed in claim 74 wherein the reinforcement comprises an inelastic element.
